(19) United States
(12) Patent Application Publication
(10) Pub. No.: US 2024/0012000 A1
(43) Pub. Date: Jan. 11, 2024

(54) SOLID MIXTURE COMPRISING STANDARD PROTEIN

(71) Applicant: ProteomEdge AB, Stockholm (SE)

(72) Inventors: Fredrik Edfors, Stockholm (SE); David Kotol, Enskede (SE); Mathias Uhlén, Lidingö (SE)

(73) Assignee: ProteomEdge AB, Stockholm (SE)

(21) Appl. No.: 18/040,411

(22) PCT Filed: Aug. 10, 2021
(86) PCT No.: PCT/EP2021/072223

§ 371 Date: Feb. 2, 2023

(30) Foreign Application Priority Data
Aug. 10, 2020 (EP) 20190220.2

(51) Int. Cl. G01N 33/68

(52) U.S. Cl. CPC G01N 33/6848; G01N 2333/46

(57) ABSTRACT

Disclosed are a mixture comprising at least one internal standard protein, a container comprising the mixture, a method for preparing a container with the mixture, a method for determining the amount of a target protein present in a sample, providing a container comprising the mixture, as well as a kit for carrying out the methods.

### TECHNICAL FIELD

The present disclosure relates to a mixture comprising at least one internal standard protein. The disclosure further relates to a container comprising the mixture, a method for preparing a container with the mixture, a method for determining the amount of a target protein present in a sample, providing a container comprising the mixture. Lastly, the present disclosure relates to a kit for carrying out any of the methods disclosed herein.

### BACKGROUND

Measurement of protein levels in body fluid is an essential component of assessing the health state of an individual. Measurement of protein levels in research samples is an essential component of understanding protein function and relevance in e.g. various cell types. A large number of proteomics technologies have successfully been established and implemented into clinical practice, capable of providing information describing patients at the molecular level. More than one hundred clinical protein assays have been approved by the US Food and Drug Administration (FDA) for use in serum or plasma, and an equally large number of targets have been cleared for standardized laboratory tests in the US.

Mass spectrometry (MS) technologies are capable of simultaneous analysis of a plurality of target proteins (multiplex), due to the high speed of the detector and the separation by mass. This is especially true when MS is used together with liquid chromatographic separation of proteins or peptides (LC-MS). Quantitative proteomics using mass spectrometry read-out provides both sensitive and robust assays when quantifying proteins from complex samples such as cell-lines, tissues and body fluids.

Targeted proteomics is a mass-spectrometry-based approach focusing on pre-defined sets of target proteins, which are measured with high reproducibility across many samples. This approach has been shown to be suitable for studies with clinical applications, where it may be advantageous to carry out multiplex analysis of a sample.

Quantitative determination of analytes by MS requires the use of a standard of known amount in the sample. Addition of standards enable intra-assay normalization between measured heavy and light peptide peaks, i.e. between peaks from peptides that are labeled with heavy isotopes vs peaks from unlabeled endogenous peptides.

The highest level of quantitative reproducibility is generally achieved when isotopically labeled standards are used. Here, internal standards are isotopically labeled either through metabolic or chemical labeling of the sample or by simple addition of stable isotope standard (SIS) peptides or proteins to the sample.

Internal standards are usually added to the sample of interest in a soluble format, but this is not always the case. WO2005/031304 describes methods of quantifying the levels of at least one analyte in a sample or extract using mass spectrometry, using at least one internal standard that may be lyophilized over the surface of the interior wall of a collection device. The internal standard is typically a dendrimer, such as a PEG dendrimer.

WO2017/210147 describes a kit for detecting biomarkers comprising at least one internal standard, which kit may be configured to be used for mass spectroscopy. The internal standard may be freeze-dried.

Proteins may suffer from poor stability if not handled with great care, due to for example denaturation or fragmentation. For applications within the proteomics field, increased ease of use of internal standards would be beneficial.

For these and other reasons, there is a need for increased stability of internal standard proteins at various conditions and upon storage. In other words, there is a call for protein mixtures with improved stability.

### Disclosure of the Invention

It is an object of the disclosure to at least partly reduce or overcome challenges in the prior art, and provide means for obtaining a stable mixture comprising at least one standard protein. It is another object of the disclosure to provide means for a one-pot system in targeted proteomics. The one-pot system enables analysis of a plurality of target proteins, such as a large cohort of target proteins.

In a first aspect of the disclosure, there is provided a solid mixture. The solid mixture comprises at least one internal standard protein, at least one chaotropic agent or derivative or salt thereof; and optionally a buffer.

Chaotropic agents are molecules that are able to disrupt the hydrogen bonding network between water molecules. Non-limiting examples of chaotropic agents, which may be useful in embodiments of the disclosure, are urea, guanidinium, thiourea, n-butanol, ethanol, lithium perchlorate, sodium perchlorate, lithium acetate, magnesium chloride, phenol, 2-propanol and sodium dodecyl sulfate. In some embodiments, the chaotropic agent is selected from the group consisting of urea, guanidine, thiourea, and derivatives and salts thereof.

As used herein, the term “derivative” may mean a similar compound or precursor compound. A “derivative” may also mean that the named compound is part of a larger structure.

Because the solid mixture of the first aspect is just that, solid, it comprises no or only a minimal amount of solvent and therefore has a very small volume compared to an aqueous or other solution of a standard protein. Advantageously, because of the small volume, the solid mixture may comprise a plurality of internal standard proteins, without this having any significant effect on the final sample volume. This, in turn, provides for easier multiplex analysis of a sample. Without wishing to be bound by theory, it has been surprisingly found that the presence of a chaotropic agent in the solid mixture of the first aspect provides benefits in that the at least one internal standard protein therein enjoys an improved stability as compared to previously known protein standard mixtures.

The obtained increased or retained stability may be increased or retained stability over time and/or increased or retained stability over fluctuations in temperature. Such beneficial effect provides for ease of storage, including long-term storage. For example, the mixture of the present invention may be stored at room temperature during transport, which may reduce transportation costs. It may also provide for a more climate friendly transportation as it does not require specific temperatures to be held. Additionally, it provides ease of use both for the manufacturer and for a user of a product comprising the solid mixture. One of many possible reasons for the improved or retained stability of the proteins in the solid mixture, is that proteins are not degraded or fragmented to the same extent as in solution.

It was surprisingly found that one may cause a protein, such as an internal standard protein, to dry out together with a chaotropic agent, and re-dissolve the protein for subsequent use at a later time. This finding contradicted expected difficulties connected to re-dissolution of proteins that had been subjected to evaporation. In effect, such proteins have been considered to be difficult to completely dissolve. The level of dissolution is of great importance in for example quantitative mass spectrometry and other proteomics applications, because it is crucial for accurate quantity determination. By using the presently disclosed solid mixture in proteomics applications, systematic errors may be minimized.

In particular, recombinantly produced protein standards were known in the art to be sensitive to buffer storage conditions and to be likely to aggregate or precipitate. A reason for this may be the formulation in which they are present, which formulation is an artefact from the recombinant production and subsequent purification. These problems are contemplated to be alleviated or avoided with the use of a chaotropic agent in the mixture.

In terms of handling, it is furthermore advantageous that the chaotropic agent may be added already during recombinant production of standard protein or during purification of synthetic standard protein. When this is the case, easy and fast production of the solid mixture as disclosed herein is achieved.

An advantage with the solid mixture, methods and kit of the various aspects of the disclosure is that there is less need for handling liquids in the workflow of analyzing samples and transporting protein standards. A problem arising when transporting a liquid in a container is that liquid is likely to be lost due to droplets forming on for example the walls or a lid of the container. Droplets may be formed by splashing of the liquid onto undesired parts of the container. In this way, when using the container, it is difficult to incorporate all of the liquid in the container, which may in turn lead to inaccurate determination when using the container for quantitative purposes. Also, the concentration of the contents in the mixture may be changed, due to condensation of liquid, potentially affecting the accuracy of measurement of the absolute quantity of the members of the mixture. This is a clear disadvantage when the container comprising standard proteins is used for quantitative measurements, for example in mass spectrometry and other proteomics methods. Some or all of these disadvantages connected to liquid handling are alleviated by use of the solid mixture of the present disclosure.

In some embodiments, the chaotropic agent in the solid mixture is selected from the group consisting of urea, guanidine, thiourea and derivatives and salts thereof.

In some embodiments, the chaotropic agent in the solid mixture is thiourea or a derivative or salt thereof. In a specific such embodiment, the chaotropic agent in the solid mixture is thiourea. In some embodiments, the chaotropic agent in the solid mixture is guanidine or a derivative or salt thereof. In a specific such embodiment, the chaotropic agent in the solid mixture is guanidine. In preferred embodiments, the chaotropic agent in the solid mixture is urea or a derivative or salt thereof. In a specific such embodiment, the chaotropic agent in the solid mixture is guanidine.

Upon addition to the mixture to be solidified, the chaotropic agent of the solid mixture may in some embodiments be present in a concentration of at least 0.25 M, such as at least 0.5 M, such as at least 1 M, such as at least 2 M, such as at least 3 M, such as at least 4 M, such as at least 5 M, such as at least 6 M, such as at least 7 M, such as at least 8 M.

An advantage of the solid mixture as disclosed herein is that when present in the mixture, the at least one internal standard protein remains stable upon storage for at least 1 week, such as at least 2 weeks, such as at least 3 weeks, such as at least 4 weeks, such as at least 5 weeks, such as at least 6 weeks, such as at least 7 weeks, such as at least 8 weeks, such as at least 9 weeks, such as at least 10 weeks, such as at least 3 months, such as at least 6 months, such as at least 1 year, such as at least 2 years.

An advantage of the solid mixture as disclosed herein is that when present in the mixture, the least one internal standard protein remains stable upon storage at a temperature of at least 4° C., such as at least 7° C., such as at least 10° C., such as at least 15° C., such as at least 20° C., such as at least 25° C., such as at least 30° C., such as at least 35° C., such as at least 40° C.

Furthermore, the at least one internal standard protein may remain stable upon storage at a temperature of at most 0° C., such as when stored at at most −10° C., such as at at most −20° C., such as at at most −50° C., such as when stored at at most −80° C.

Retained stability during temperature fluctuations improves the ease of use of the presently disclosed solid mixture. In some embodiments, the at least one internal standard protein remains stable when subjected to at least 1 freeze-thaw cycle, such as at least 2 freeze-thaw cycles, such as at least 3 freeze-thaw cycles, such as at least 4 freeze-thaw cycles, such as at least 5 freeze-thaw cycles, such as at least 6 freeze-thaw cycles, such as at least 7 freeze-thaw cycles, such as at least 8 freeze-thaw cycles, such as at least 9 freeze-thaw cycles, such as at least 10 freeze-thaw cycles, such as at least freeze-thaw cycles, such as at least 20 freeze-thaw cycles.

In related embodiments, the stability of at least one internal standard protein is retained upon fluctuating temperatures. That is, it may not be strictly necessary to store the at least one internal standard protein at a specific temperature. It is expected that the protein(s) are stable even when change in temperature occurs. Fluctuations may be a variation of up to over 10° C., such as over 20° C., such as over 30° C., such as over 40° C., such as over 50° C., such as over 60° C., such as over 70° C., such as over 80° C., such as over 90° C.

As used herein, the term “retained stability” of a protein is intended to mean that unwanted phenomena such as irreversible aggregation, degradation or fragmentation of the protein do not occur, i.e. that the ability to renature the protein from a denatured state to a to a non-aggregated and non-degraded form, wherein the protein is susceptible to cleavage, with e.g. trypsin or another proteolytic protein, is retained. Stability is preferably determined by a coefficient of variation (“CV”). A skilled person realizes that a low measure of variation between samples (e.g. in a series of measurements over time) signifies a higher degree of retained stability. In one embodiment, “retained stability” means that the CV exhibited upon comparison of different samples is at most about 20%, such as at most 15%, such as at most 10%. As known to a person of skill in the art, other ways of measuring and denoting retained or increased stability are also possible.

Non-limiting examples of methods to determine the stability of proteins are bottom-up proteomics, top-down proteomics or immuno-affinity enrichment followed by either colorimetric read-out or LC-MS/MS. Non-limiting examples of methods to determine if a protein is aggregated are SDS-PAGE and mass spectrometry.

In the field of proteomics, retained stability furthermore translates into retained quantitative accuracy and precision over time. As used herein, retained stability of a protein means that quantification of the same protein yields the same result, or at least a result within the same range, at two different time points. In analogy with the discussion above, a “result within the same range” typically involves a coefficient of variation of at most 20%, such as at most 15%, such as at most 10%. Optionally, between the different time points, which time points may be separated by a long period of time, one or more freeze-thaw cycles have been carried out, or the solid mixture has been stored at one or more specific temperatures.

An advantage of the solid mixture as disclosed herein is that the mixture may comprise more than one standard protein, such as a plurality of standard proteins. In some embodiments, the at least one internal standard protein is at least 2 standard proteins, such as at least 5 standard proteins, such as at least 10 standard proteins, such as at least 20 standard proteins, such as at least 30 standard proteins, such as at least 40 standard proteins, such as at least 50 standard proteins, such as at least 60 standard proteins, such as at least 70 standard proteins, such as at least 80 standard proteins, such as at least 90 standard proteins, such as at least 100 standard proteins, such as at least 200 standard proteins, such as at least 300 standard proteins, such as at least 400 standard proteins, such as at least 500 standard proteins.

It may be advantageous that the standard protein in the solid mixture comprises a label in order to be distinguished from a naturally occurring protein, such as a target protein. Labels that can be used are known to a person of skill in the art, and may be selected from the group consisting of stable isotope labeled amino acids enriched with heavy isotopes or any other enriched isotope. In preferred embodiments, the internal standard protein comprises an isotopic label. In some embodiments, the internal standard protein comprises at least one isotopically labeled amino acid. In some embodiments, the isotopic label is selected from the group consisting of ¹⁵N, ¹³C and ¹⁸O.

It is well known in the art how to produce a protein. A protein may for example be produced by means of recombinant DNA technology, or may be produced by means of a peptide synthesizer. In some embodiments, the internal standard protein is a recombinant protein. In other embodiments, the internal standard protein is a synthetic protein.

In some embodiments, the mixture comprising said at least one internal standard protein and at least one chaotropic agent further comprises phosphate, or another substance with buffering properties. The phosphate may originate from a buffer in which the internal standard may be stored before use. As apparent to persons of skill in the art, the buffer can be any buffer suitable for buffering protein-comprising solutions. Thereby, in other embodiments, the mixture comprising said at least one internal standard protein and at least one chaotropic agent further comprises another substance with buffering properties. If the buffer comprises phosphate, the buffer can be any phosphate buffer, such as phosphate buffer saline (PBS). Buffers comprising another compound with buffering properties may be one of the following non-limiting examples: Tris, HEPES, MOPS, MES, PIPES and ABC (ammonium bicarbonate). Suitable molarity of the buffer, as well as pH and any additives, can be determined by a person of skill in the art.

In some embodiments, the solid mixture may further comprise a sample suspected of comprising at least one target protein. As a non-limiting list of examples, said sample may be a bodily fluid sample, a cell sample or a tissue sample. The skilled person is aware of other types of samples that comprise proteins and could also be used. Non-limiting examples of bodily fluid samples suitable for use in the solid mixture as disclosed herein are plasma, serum, blood, cerebrospinal fluid, dry blood spots, saliva and urine. In preferred embodiments, the sample is a bodily fluid sample selected from the group consisting of plasma, serum, blood, cerebrospinal fluid, dry blood spots and saliva. In order to store the solid mixture further comprising said sample, it is preferred that there is no liquid in the mixture formed. In some embodiments, the sample is solidified.

In some embodiments, the internal standard protein comprises a fragment of said target protein. In other embodiments, the internal standard protein is the full length target protein, except that it comprises a label in addition. Contemplated labels are discussed above.

In some embodiments, the solid mixture is suitable for use in mass spectrometry. The type of mass spectrometry used may for example be tandem mass spectrometry with data dependent acquisition mode, tandem mass spectrometry with data independent acquisition mode or tandem mass spectrometry with selective reaction monitoring mode. As apparent to a person of skill in the art, other types of mass spectrometry methods are also possible to use. The mass analyzer of the mass spectrometry instrument may be an ion trap, a triple quadrupole, an ESI-TOF, a Q-TOF type instrument, an orbitrap, or any other instrument of suitable mass resolution (>1,000) and sensitivity.

In some embodiments, the solid mixture is suitable for use in proteomics, such as targeted proteomics.

In a second aspect of the disclosure, there is provided a method for preparing a container comprising a solid mixture comprising at least one internal standard protein. The method comprises the steps of providing a solution comprising the at least one internal standard protein and at least one chaotropic agent or derivative or salt thereof, placing the solution in a container and removing residual liquid from said solution. Thereby, a container comprising a solid mixture is obtained. The solid mixture comprises the at least one internal standard protein and the at least one chaotropic agent.

In some embodiments, the chaotropic agent is selected from the group consisting of urea, guanidine, thiourea and derivatives and salts thereof. In some embodiments, the chaotropic agent is guanidine or a derivative or salt thereof. In some embodiments, the chaotropic agent is thiourea or a derivative or salt thereof. In preferred embodiments, the chaotropic agent is urea or a derivative or salt thereof.

In some embodiments, the chaotropic agent is present in the solution in a concentration of at least 0.25 M, such as at least 0.5 M, such as at least 1 M, such as at least 2 M, such as at least 3 M, such as at least 4 M, such as at least 5 M, such as at least 6 M, such as at least 7 M, such as at least 8 M.

In some embodiments, the solution comprising said at least one internal standard protein and at least one chaotropic agent further comprises phosphate or another substance with buffering properties. Any suitable substance with buffering properties may be comprised in the solution, as discussed above in relation to the first aspect of the disclosure.

In some embodiments, the step of removing residual liquid from the solution comprises removing liquid by means of reduced pressure. In preferred embodiments, the step of removing liquid by means of reduced pressure is by means of vacuum drying. In addition to applying vacuum, it is advantageous to also apply heat. In some embodiments, the step of removing liquid by means of vacuum is performed at a temperature of 5-60° C., such as at 10-50° C., such as at 15-45° C., such as at 20-45° C., such as at 25-45° C., such as at 30-45° C., such as at 35-45° C., such as 40-45° C., such as at 42° C. Application of heat decreases the time it takes to remove residual liquid from the solution in order to obtain a solid mixture.

When removing liquid by means of reduced pressure, it is also possible to use means of freeze drying.

An advantage of the method of the second aspect of the disclosure is that it provides for retained stability of the at least one internal standard protein upon storage. Storage may be for at least 1 week, such as at least 2 weeks, such as at least 3 weeks, such as at least 4 weeks, such as at least 5 weeks, such as at least 6 weeks, such as at least 7 weeks, such as at least 8 weeks, such as at least 9 weeks, such as at least 10 weeks, such as at least 3 months, such as at least 6 months, such as at least 1 year, such as at least 2 years.

An advantage of the method of the second aspect of the disclosure is that it provides for retained stability of the at least one internal standard protein upon storage at a temperature of at least 4° C., such as at least 7° C., such as at least 10° C., such as at least 15° C., such as at least 20° C., such as at least 25° C., such as at least 30° C., such as at least 35° C., such as at least 40° C.

In some embodiments, the method disclosed herein provides for retained stability of the at least one internal standard protein upon storage at a temperature of at most 0° C., such as stored at at most −10° C., such as stored at at most −20° C., such as stored at at most −50° C., such as stored at at most −80° C. In some related embodiments, the stability of at least one internal standard protein is retained upon fluctuating temperatures, as discussed above in relation to the first aspect of the disclosure.

Furthermore, it is expected that the retained stability is provided also for repeated fluctuation in temperature. In some embodiments, the method provides retained stability when subjected to at least 1 freeze-thaw cycle, such as at least 2 freeze-thaw cycles, such as at least 3 freeze-thaw cycles, such as at least 4 freeze-thaw cycles, such as at least 5 freeze-thaw cycles, such as at least 6 freeze-thaw cycles, such as at least 7 freeze-thaw cycles, such as at least 8 freeze-thaw cycles, such as at least 9 freeze-thaw cycles, such as at least 10 freeze-thaw cycles, such as at least 15 freeze-thaw cycles, such as at least 20 freeze-thaw cycles, such as at least 50 freeze-thaw cycles.

In some embodiments, the retained stability is determined by a coefficient of variation, as discussed above in relation to the first aspect of the disclosure.

An advantage of the method of the second aspect is that a plurality of internal standard proteins may be added to the same container. In this way, the container may be manufactured to comprise a plurality of internal standard proteins. In some embodiments, the at least one internal standard protein is at least 2 standard proteins, such as at least 5 standard proteins, at least 10 standard proteins, such as at least 20 standard proteins, such as at least 30 standard proteins, such as at least 40 standard proteins, such as at least 50 standard proteins, such as at least 60 standard proteins, such as at least 70 standard proteins, such as at least 80 standard proteins, such as at least 90 standard proteins, such as at least 100 standard proteins, such as at least 200 standard proteins, such as at least 300 standard proteins, such as at least 400 standard proteins, such as at least 500 standard proteins. Thereby, a large cohort of target proteins can be analyzed simultaneously within one sample without diluting the sample volume to any significant extent.

In an alternative embodiment, the container may be manufactured to comprise one internal standard protein for use in single-plex analysis of at least one target protein, such as at least 5 target proteins, such as at least 10 target proteins, such as at least 20 target proteins, such as at least 30 target proteins, such as at least 40 target proteins, such as at least 50 target proteins, such as at least 60 target proteins, such as at least 70 target proteins, such as at least 80 target proteins, such as at least 90 target proteins, such as at least 100 target proteins, such as at least 200 target proteins, such as at least 300 target proteins, such as at least 400 target proteins, such as at least 500 target proteins.

As discussed above in relation to the first aspect of the disclosure, it may be advantageous if the internal standard protein comprises a label in order to be distinguished from a natural protein, such as a target protein.

Examples of labels are discussed above. In preferred embodiments, the internal standard protein comprises an isotopic label. In some embodiments, the internal standard protein comprises at least one isotopically labeled amino acid. In preferred embodiments, the isotopic label is selected from the group consisting of ¹⁵N, ¹³C and ¹⁸O.

As discussed above in relation to the first aspect of the disclosure, a protein may for example be produced by means of recombinant DNA technology, or may be produced by means of a peptide synthesizer. In some embodiments, the internal standard protein is a recombinant protein. In other embodiments, the internal standard protein is a synthetic protein.

In a third aspect of the disclosure, there is provided a container comprising a solid mixture according to any embodiment of the first aspect. One advantage of such a container is that the mixture is maintained in the container and may be fixed to the bottom of the container by virtue of being a solid. In this way, higher accuracy when determining the quantity of the members of the mixture and/or present in an added sample is enabled.

Furthermore, the third aspect provides a container prepared using the method according to any embodiment of the second aspect. The container according to the third aspect of the disclosure may be selected from the group consisting of a microtiter plate, a vial, a collection tube, a bottle, a pre-coated filter paper, a blood tube, a Whatman paper, a DBS collection device, a dried plasma spot device, a dried serum spot device and a culturing plate. Other types of containers are also plausible, as apparent to persons of skill in the art. In some embodiments, the container is suitable for use in mass spectrometry. In some embodiments, the container is suitable for use in proteomics.

In a fourth aspect of the disclosure, there is provided a method for determining the amount of a target protein present in a sample. The method comprises the steps of providing a container according to the third aspect of the disclosure. Depending on which embodiment of the foregoing aspects that was used in preparing the container, it may or may not comprise a sample. If it does not already comprise a sample, such a sample is added in a step of the method of this aspect. As a non-limiting list of examples, said sample may be a bodily fluid sample, a cell sample or a tissue sample. The skilled person is aware of other types of samples that comprise proteins and could also be used. Whether added beforehand when preparing the container or added in connection with carrying out the method of the fourth aspect, the end result is that a sample is included in said mixture, thereby constituting a test sample. The method further comprises the steps of subjecting the test sample to analysis and using the results of the analysis to determine the amount of the at least one target protein in the sample by comparison with said internal standard protein.

In some embodiments of the fourth aspect, the internal standard protein comprises a fragment of said target protein. In other embodiments, the internal standard protein is the full length target protein except in a label, as discussed above.

In some embodiments, the determination of the amount of the at least one target protein is performed using mass spectrometry.

In some embodiments, the method further comprises evaporating said sample. If evaporated, the method may further comprise a step of long-term storage of the sample. The long-term storage occurs before subjecting the sample to analysis and subsequent determination of the amount of the at least one target protein in the sample by comparison with the standard protein. In some embodiments, the long-term storage is for at least 1 week, such as at least 2 weeks, such as at least 3 weeks, such as at least 4 weeks, such as at least 5 weeks, such as at least 6 weeks, such as at least 7 weeks, such as at least 8 weeks, such as at least 9 weeks, such as at least 10 weeks, such as at least 3 months, such as at least 6 months, such as at least 1 year, such as at least 2 years.

In some embodiments, the sample is a bodily fluid sample selected from the group consisting of plasma, serum, blood, cerebrospinal fluid, dry blood spots, saliva and urine.

As apparent to the skilled person, some methods of analysis, e.g. mass spectrometry, require that a solid mixture according to the disclosure is first dissolved or reconstituted in a suitable liquid or a fluid sample before analysis can be carried out.

In a fifth aspect of the disclosure, there is provided a kit for carrying out the method according to any of the disclosures of the fourth aspect of the disclosure. The kit comprises a container according to the third aspect of the disclosure and instructions for carrying out the method.

### BRIEF DESCRIPTION OF THE DRAWINGS

FIG. 1 schematically shows that the tested isotopically labeled internal protein standards represent a stretch of amino acids which is unique for the target protein of interest, and are fused to a tag sequence, denoted “Tag-heavy”, which is used for quantification of the internal standard protein by comparison with an identical sequence, denoted “Tag-light”, which is not isotopically labelled.

FIG. 2 illustrates the workflow used to estimate the effect of vacuum drying internal standard proteins as compared to internal standard proteins kept in solution, as well as the effect of room temperature storage on the stability of vacuum-dried internal standard proteins.

FIG. 3 shows extracted chromatograms, showing overlaps of the areas under the curve of a peptide resulting from trypsin digestion of the Tag-heavy and Tag-light polypeptide sequences.

FIG. 4 shows the result of a comparison of median values from triplicate digestion and Tag-based quantification of isotopically labeled standard proteins that were kept in solution and of isotopically labeled standard proteins that were vacuum dried according to the present disclosure.

FIG. 5 shows a density plot of CVs between the quantification results of all vacuum dried isotopically labeled protein standards stored at room temperature for 0 (median of the triplicate), 1 and 4 weeks, as illustrated in FIG. 2.

FIG. 6 illustrates the workflow used to estimate the quantification precision of 100 proteins subjected to different digestion times, using a mixture of 100 vacuum dried internal standard proteins.

FIG. 7 shows the results of cluster analysis, exhibiting the same digestion efficiency for both endogenous proteins and internal standard proteins according to the disclosure for most peptides, i.e. the peptides of Cluster 2 and Cluster 4.

FIG. 8 shows the technical reproducibility of quantification as coefficient of variation between three technical replicates per peptide of every time point with medians (indicated in the figure) ranging from 4.6% to 6.1%.

FIG. 9 shows all quantified proteins using the mixture of vacuum dried internal standard proteins and their dynamic range, as measured after 16 hours of digestion.

### EXAMPLES

### Example 1

### Stability Assessment of Vacuum-Dried, Isotopically Labeled Protein Standards

### Materials and Methods

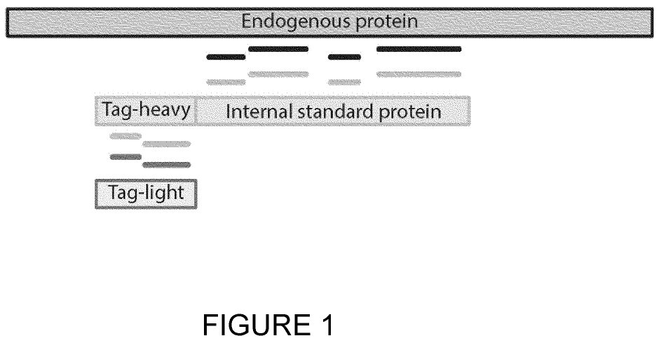

96 internal standard proteins were randomly selected from an in-house produced library of stable isotope-labeled internal standard proteins, and aliquots thereof were individually added to a 96-well plate. Subsequently, quantification tag (“Tag-light”, absolutely quantified by amino acid analysis) was diluted to a final concentration of 10 μM in 1x PBS (phosphate buffered saline) and 1 M urea, and aliquoted to another 96-well plate. FIG. 1 illustrates the relationship between the endogenous protein, the internal standard protein, the Tag-heavy sequence and the Tag-light sequence. Aliquots from the plate with internal standard proteins were distributed into 8 new plates, so that every well contained 5 μl (˜50 pmol) of each internal standard protein. Five of the eight plates were vacuum dried at 42° C. for 3 h and stored at room temperature. During that time, the remaining three plates were kept on ice with the internal standard proteins in solution.

Digestion: Three of the five vacuum dried plates were prepared together with the three plates of aliquoted standards kept in solution. Prior to the addition of 5 μl of 10 μM Tag-light, 55 μl of 90.9 mM ammonium bicarbonate (ABC) were added to each well of the 96-well plates with vacuum dried internal standard proteins and 50 μl of 100 mM ABC were added to each well of the 96-well plates with internal standard proteins that were kept in solution. All six plates were sonicated for 180 s and digested by addition of 200 ng of porcine trypsin (Thermo Scientific) overnight at 37° C. Digestion was quenched by addition of formic acid (FA) to a final concentration of 0.6% (v/v) and samples were analyzed using LC-MS/MS operating in parallel reaction monitoring (PRM) mode.

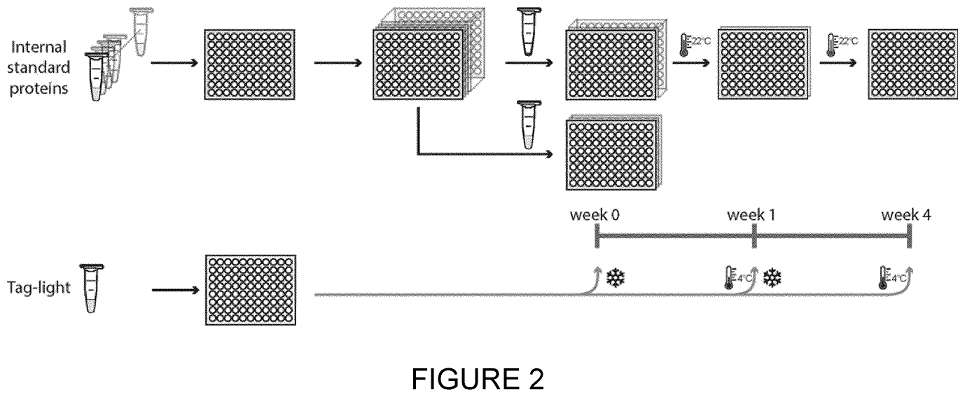

The remaining two of the five vacuum dried plates were kept at room temperature for 1 and 4 weeks, respectively, prior to addition of ABC and Tag-light, trypsin digestion and LC-MS/MS-PRM analysis as described above. The workflow is illustrated in FIG. 2.

Quantification of Internal Standard Proteins Usinq LC-PRM:

Quantification was performed using an Ultimate 3000 LC online system (Thermo Fisher) connected to Q Exactive HF MS (Thermo Fisher). 2.5 pmol of each internal standard protein was loaded onto an Acclaim PepMap 100 trap column (cat. no. 164535; Thermo Scientific), washed 3 min at 8.5 μl/min with Solvent A (3% acetonitrile (ACN), 0.1% FA and then separated by an analytical PepMap RSLC C18 column (cat. no. ES802; Thermo Scientific). A linear 3-min gradient ranging from 3% to 20% Solvent B (95% ACN, 0.1% FA) at 0.6 μl/min was used for eluting the peptides. The analytical column was then washed for 3 min at 1 μl/min with 99% solvent B and re-equilibrated with 3% B at 0.6 μl/min for 4 min.

The MS operated in PRM mode with each cycle comprising one full MS scan performed at 15,000 resolution (AGC target 2e5, mass range 350-1,600 m/z and injection time 55 ms) followed by 20 PRM MS/MS scans at 15,000 resolution (AGC target 1e6, NCE 27, isolation window 1.5 m/z and injection time 105 ms) defined by a scheduled (0.4 min windows) isolation list.

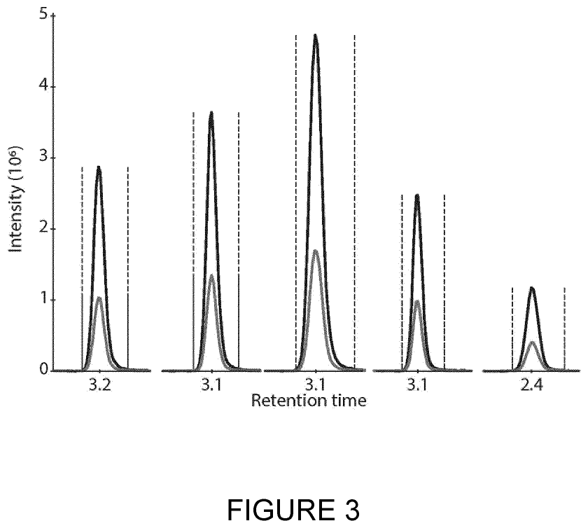

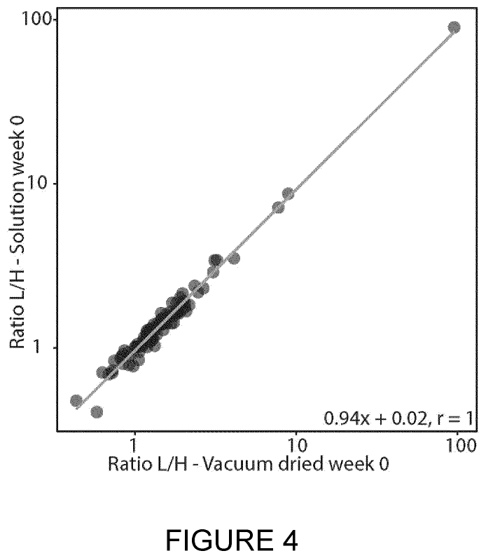

Resulting RAW files were loaded into Skyline (v. 20.1.0.76; MacLean et al. (2010), Bioinformatics 26:966-968) together with sequences and transitions of Tag-light and Tag-heavy. Chromatograms for transitions of peptide DLQAQWESAK (SEQ ID NO:1; Table 1) were extracted from the RAW files and areas under the respective curves calculated as shown in FIG. 3 for five examples. The ratios between heavy and light Tag sequence peptides were exported, and the ratios obtained with standard proteins in solution were plotted against those obtained using vacuum dried internal standard proteins according to the present disclosure (FIG. 4).

### Results

The ratios of areas under the curve for light and heavy Tag peptides obtained using the isotopically labeled internal standard proteins that were kept in solution were plotted against the corresponding ratios obtained using isotopically labeled internal standard proteins that had been vacuum dried (FIG. 4). The results demonstrate that there is no difference between the two strategies and that the amount of accessible, isotopically labeled standard protein is the same, whether the isotopically labelled standard was kept in solution or vacuum dried and solid prior to addition of sample to be analyzed and enzymatic digestion.

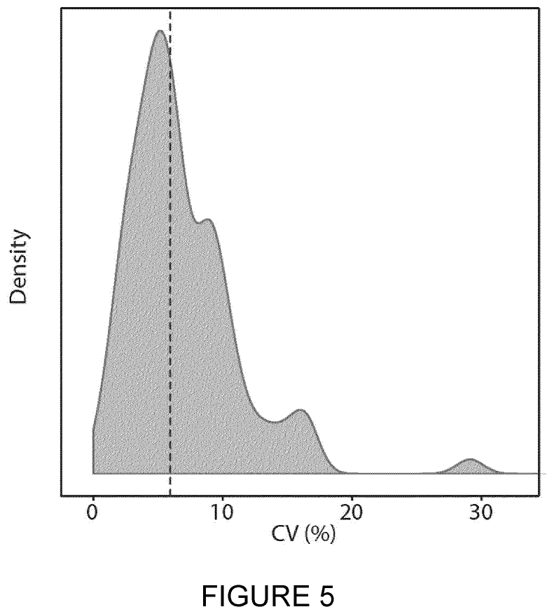

Furthermore, plates stored at room temperature over extended periods of time in the vacuum dried format (1 and 4 weeks) showed a retained stability and accessibility of the isotopically labeled protein standards, as evidenced by the coefficients of variation (CVs) below 20%. This is shown in FIG. 5. Only one isotopically labeled standard protein exhibited a CV above 20% (CV=29.1%). For this variant, the internal standard protein and its Tag-heavy sequence were 100 times less abundant than the added Tag-light quantification tag, which caused the decreased precision of quantification and suggests that production of this isotopically labeled standard protein wasn't entirely successful. In fact, the CV of triplicate quantification of the same isotopically labeled standard protein kept in solution was 36.8% (data not shown), which supports the hypothesis that the decreased precision of quantification between the three time points was caused by the big difference in amount (large off-ratio) between the Tag-light and the Tag-heavy peptides and that the accessible amount of this internal standard protein remained the same.

Decay of the Tag-light quantification tag due to repeated freeze-thaw cycles was observed, but was constant over all replicates and could therefore be normalized for. The normalization for constant Tag-light degradation over the three repeated freeze-thaw cycle is reasonable, since it is not possible for the amount of internal standard protein to increase over time.

TABLE 1
List of transitions used for quantification within LC-PRM analysis

| Peptide Modified | Precursor | Precursor | Product | Product | Fragment |
|---|---|---|---|---|---|
| Sequence | Mz | Charge | Mz | Charge | lon |
| DLQAQVVESAK (light) | 594.317 | 2 | 632.361367 | 1 | y6 |
| DLQAQVVESAK (light) | 594.317 | 2 | 533.292953 | 1 | y5 |
| DLQAQVVESAK (light) | 594.317 | 2 | 434.224539 | 1 | y4 |
| DLQAQVVESAK (light) | 594.317 | 2 | 305.181946 | 1 | y3 |
| DLQAQVVESAK (light) | 594.317 | 2 | 218.149918 | 1 | y2 |
| DLQAQVVESAK (light) | 594.317 | 2 | 229.118283 | 1 | b2 |
| DLQAQVVESAK (light) | 594.317 | 2 | 357.176861 | 1 | b3 |
| DLQAQVVESAK (light) | 594.317 | 2 | 428.213974 | 1 | b4 |
| DLQAQVVESAK (light) | 594.317 | 2 | 556.272552 | 1 | b5 |
| DLQAQVVESAK (light) | 594.317 | 2 | 655.340966 | 1 | b6 |
| DLQAQVVESAK (heavy) | 598.3241 | 2 | 640.375566 | 1 | y6 |
| DLQAQVVESAK (heavy) | 598.3241 | 2 | 541.307152 | 1 | y5 |
| DLQAQVVESAK (heavy) | 598.3241 | 2 | 442.238738 | 1 | y4 |
| DLQAQVVESAK (heavy) | 598.3241 | 2 | 313.196145 | 1 | y3 |
| DLQAQVVESAK (heavy) | 598.3241 | 2 | 226.164117 | 1 | y2 |
| DLQAQVVESAK (heavy) | 598.3241 | 2 | 229.118283 | 1 | b2 |
| DLQAQVVESAK (heavy) | 598.3241 | 2 | 357.176861 | 1 | b3 |
| DLQAQVVESAK (heavy) | 598.3241 | 2 | 428.213974 | 1 | b4 |
| DLQAQVVESAK (heavy) | 598.3241 | 2 | 556.272552 | 1 | b5 |
| DLQAQVVESAK (heavy) | 598.3241 | 2 | 655.340966 | 1 | b6 |

### Example 2

### Quantification of Plasma Proteins Using Vacuum Dried Internal Standard Proteins

### Materials and Methods

100 isotopically labeled internal standard proteins (in 1 M urea and 1x PBS) targeting 100 human endogenous plasma proteins were mixed in a single container. The mixture was aliquoted to 15 tubes and vacuum dried for 3 hours at 42° C. Sodium deoxycholate (SDC) was diluted in Milli-Q water and added to vacuum dried, isotopically labeled standards so that the final concentrations of SDC, urea and PBS after addition of diluted plasma were 1% SDC, 1 M urea and 1x PBS.

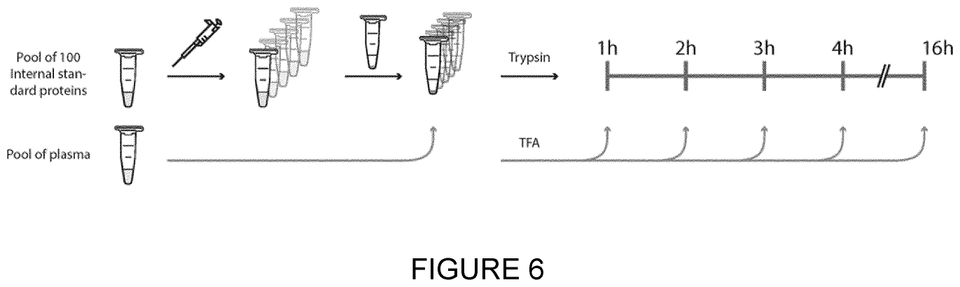

A pool of plasma from human subjects (3 males, 2 females) was diluted 10 times with 1x PBS. An amount corresponding to 0.5 μl of undiluted plasma was added into each of the 15 tubes comprising the vacuum dried mixture of internal standard proteins. Samples were treated in 10 mM DTT at 37° C. for 1 h and 50 mM CAA for 30 minutes at room temperature in the dark. SDC was diluted to a final concentration of 0.25% (w/w) with 1x PBS prior to addition of porcine trypsin (Thermo Scientific) in an enzyme:substrate ratio of 1:50. Digestion was performed at 37° C. and quenched with 0.5% (v/v) trifluoroacetic acid (TFA) after 1, 2, 3, 4 and 16 hours (FIG. 6). Quenched samples were centrifuged at 13,200 rcf for 5 min, and supernatants desalted on 3-layer C18 StageTips prepared in house (Rappsilber et al. (2007), Nat. Protoc. 2:1896-1906). In brief, StageTips were activated with 50 μl of 100% ACN and equilibrated with 50 μl 0.1% TFA followed by addition of the digested sample corresponding to 15 ug of proteins in raw plasma. The C18 matrix was washed twice with 0.1% TFA and peptides eluted in two steps with 80% ACN, 0.1% TFA. Eluted peptides were vacuum dried at 42° C. Desalted samples were dissolved in Solvent A and an amount corresponding to 4 μg protein in undiluted plasma was subjected to LC-MS/MS analysis using data-independent acquisition (DIA).

Quantification of internal standard proteins usinq LC-DIA: Analysis was performed using an Ultimate 3000 LC online system (Thermo Fisher) connected to a Q Exactive HF MS (Thermo Fisher). First, an amount corresponding to 4 μg protein in undiluted plasma was loaded onto a trap column (cat. no. 160438, Thermo Scientific) and washed for 1 min at a flow rate of 15 μl/min with Solvent A. Peptides were then separated by a 15 cm analytical column (cat. no. ES806A, Thermo Scientific). A 50 min method with a linear gradient was used for eluting the peptides, ranging from 1% to 32% Solvent B at a flow rate of 3.6 μl/min. The analytical column was washed with 99% Solvent B for 30 s followed by two seesaw gradients from 1% to 99% Solvent B. Column was then re-equilibrated for 1 min with 1% Solvent B.

The MS operated in DIA mode with each cycle comprising of one full MS scan performed at 60,000 resolution (AGC target 3e6, mass range 300-1,200 m/z and injection time 105 ms) followed by 30 DIA MS/MS scans at 30,000 resolution (AGC target 1e6, NCE 26, isolation window 12 m/z, injection time 55 ms), defined by an inclusion list ranging from 350 to 1,000 m/z. Resulting RAW files were loaded into Skyline (v. 20.1.0.76; MacLean et al. (2010), Bioinformatics 26:966-968) and ratios between areas under the curves for heavy peptides from internal protein standards and peptides from endogenous proteins were exported and analyzed.

### Results

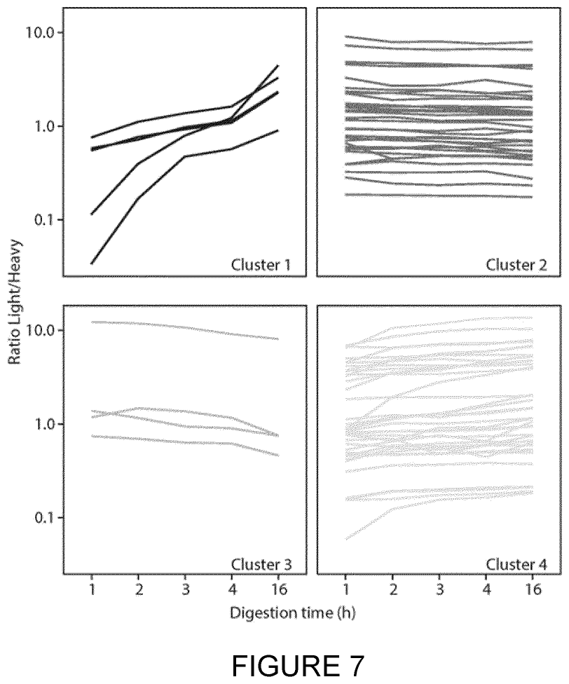

A cluster analysis was performed using the exported peptide ratios from the endogenous proteins (light) and isotopically-labelled standard proteins (heavy) at the five time points, resulting in identification of four clusters of peptides (FIG. 7). The two clusters having by far the most members (clusters 2 and 4) exhibit very little variation over time, showing that the digestion efficiency of both isotopically labelled protein standards and the corresponding endogenous proteins in corresponding peptide regions is constant throughout the time course. Cluster 1 shows, for the few members of that cluster, that there is a higher efficiency in digestion of the internal standard protein than of the endogenous protein during the time course. This results in higher amounts of certain peptides from the internal standard protein than of the corresponding peptides from the endogenous protein, as shown by the positive slope of the line before an equilibrium is reached after 16 hours. On the other hand, the few members of cluster 3 exhibit a higher efficiency for the digestion of the endogenous protein than of the isotopically labelled protein standard.

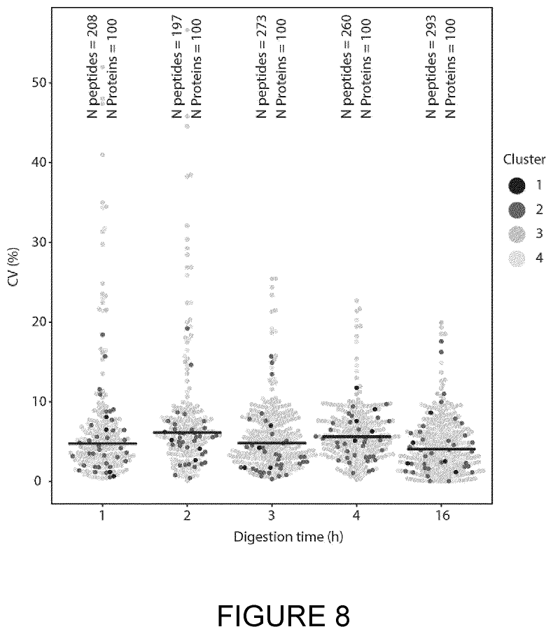

Most importantly, regardless of where the quantified peptide clusters and regardless of the digestion time, the precision of quantification remains stable and high for all clusters, with median CVs ranging between 4.6% and 6.1% (FIG. 8). This allows for short digestion times and rapid sample preparation protocols with great precision in quantification.

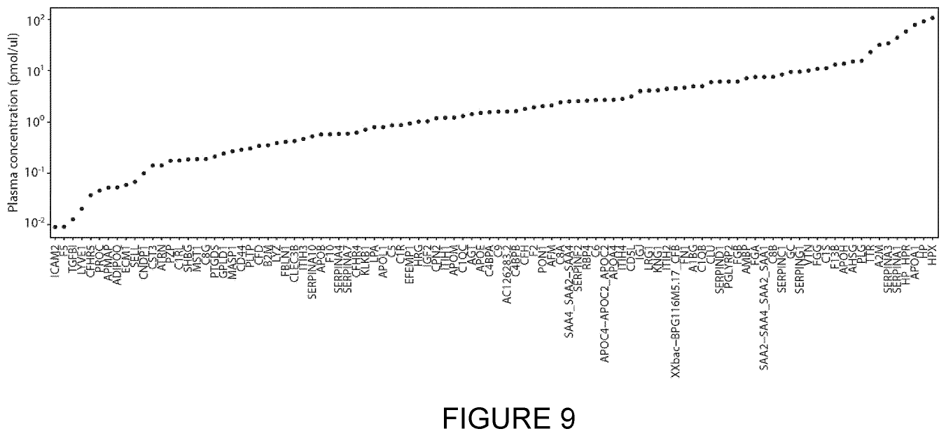

A set of 100 blood plasma proteins was quantified using a mixture of 100 internal standard proteins that was vacuum dried according to the present disclosure. The proteins were quantified using 292 peptides and cover a plasma concentration span of more than 4 orders of magnitude (10⁻²-10², FIG. 9). The median CV between the technical replicates was 4.6%, demonstrating a great precision in the assay developed.

### ITEMIZED LISTING OF EMBODIMENTS

1. Solid mixture comprising:
at least one internal standard protein;
at least one chaotropic agent or a derivative or salt thereof; and
optionally a buffer.

2. Solid mixture according to item 1, wherein said chaotropic agent is selected from the group consisting of urea, guanidine, thiourea and derivatives and salts thereof.

3. Solid mixture according to item 1, wherein said chaotropic agent is urea or a derivative or salt thereof.

4. Solid mixture according to item 3, wherein said chaotropic agent is urea.

5. Solid mixture according to item 1, wherein said chaotropic agent is guanidine or a derivative or salt thereof.

6. Solid mixture according to item 1, wherein said chaotropic agent is thiourea or a derivative or salt thereof.

7. Solid mixture according to any one of the preceding items, wherein said at least one internal standard protein remains stable upon storage for at least 1 week, such as at least 2 weeks, such as at least 3 weeks, such as at least 4 weeks, such as at least 5 weeks, such as at least 6 weeks, such as at least 7 weeks, such as at least 8 weeks, such as at least 9 weeks, such as at least weeks, such as at least 3 months, such as at least 6 months, such as at least 1 year, such as at least 2 years.

8. Solid mixture according to any one of the preceding items, wherein said at least one internal standard protein remains stable upon storage at a temperature of at least 4° C., such as at least 7° C., such as at least 10° C., such as at least 15° C., such as at least 20° C., such as at least 25° C., such as at least 30° C., such as at least 35° C., such as at least 40° C.

9. Solid mixture according to any one of the preceding items, wherein said at least one internal standard protein remains stable upon storage at a temperature of at most 0° C., such as when stored at at most −10° C., such as at most −20° C., such as when stored at at most −50° C., such as when stored at at most −80° C.

10. Solid mixture according to any one of the preceding items, wherein said at least one internal standard protein remains stable when subjected to at least 1 freeze-thaw cycle, such as at least 2 freeze-thaw cycles, such as at least 3 freeze-thaw cycles, such as at least 4 freeze-thaw cycles, such as at least 5 freeze-thaw cycles, such as at least 6 freeze-thaw cycles, such as at least 7 freeze-thaw cycles, such as at least 8 freeze-thaw cycles, such as at least 9 freeze-thaw cycles, such as at least 10 freeze-thaw cycles, such as at least freeze-thaw cycles, such as at least 20 freeze-thaw cycles.

11. Solid mixture according to any one of items 7-10, wherein said stability is determined by a coefficient of variation.

12. Solid mixture according to any one of the preceding items, wherein said at least one internal standard protein is at least 2 standard proteins, such as at least 5 standard proteins, such as at least 10 standard proteins, such as at least 20 standard proteins, such as at least 30 standard proteins, such as at least 40 standard proteins, such as at least 50 standard proteins, such as at least 60 standard proteins, such as at least 70 standard proteins, such as at least 80 standard proteins, such as at least 90 standard proteins, such as at least 100 standard proteins, such as at least 200 standard proteins, such as at least 300 standard proteins, such as at least 400 standard proteins, such as at least 500 standard proteins.

13. Solid mixture according to any one of the preceding items, wherein said internal standard protein comprises an isotopic label.

14. Solid mixture according to any one of the preceding items, wherein said internal standard protein comprises at least one isotopically labeled amino acid.

15. Solid mixture according to any one of items 13-14, wherein said isotopic label is selected from the group consisting of ¹⁵N, ¹³C and ¹⁸O.

16. Solid mixture according to any one of the preceding items, wherein said internal standard protein is a recombinant protein.

17. Solid mixture according to any one of the items 1-15, wherein said internal standard protein is a synthetic protein.

18. Solid mixture according to any of the preceding items, wherein said mixture comprising said at least one internal standard protein and at least one chaotropic agent further comprises phosphate.

19. Solid mixture according to any one of the preceding items, further comprising a sample suspected to comprise at least one target protein.

20. Solid mixture according to item 19, wherein said sample is a bodily fluid sample selected from the group consisting of plasma, serum, blood, cerebrospinal fluid, dry blood spots, saliva and urine.

21. Solid mixture according to any one of items 19-20, wherein said sample is solidified.

22. Solid mixture according to any one of items 19-21, wherein said internal standard protein comprises a fragment of said target protein.

23. Solid mixture according to any one of the preceding items, for use in mass spectrometry.

24. Solid mixture according to any one of the preceding items, for use in proteomics.

25. Method for preparing a container comprising a solid mixture comprising at least one internal standard protein, the method comprising:
providing a solution comprising said at least one internal standard protein and at least one chaotropic agent selected from the group consisting of urea, guanidine and derivatives and salts thereof,
placing said solution in a container,
removing residual liquid from said solution,
   thereby obtaining a container comprising a solid mixture comprising said at least one internal standard protein and said at least one chaotropic agent.

26. Method according to item 25, wherein said chaotropic agent is selected from the group consisting of urea, guanidine, thiourea and derivatives and salts thereof.

27. Method according to any one of items 25-26, wherein said chaotropic agent is urea or a derivative or salt thereof.

28. Method according to any one of items 25-26, wherein said chaotropic agent is guanidine or a derivative or salt thereof.

29. Method according to any one of items 25-26, wherein said chaotropic agent is thiourea or a derivative or salt thereof.

30. Method according to any one of items 25-29, wherein said chaotropic agent is present in said solution in a concentration of at least 0.5 M, such as at least 1 M, such as at least 2 M, such as at least 3 M, such as at least 4 M, such as at least 5 M, such as at least 6 M, such as at least 7 M, such as at least 8 M.

31. Method according to any one of items 25-30, wherein said solution comprising said at least one internal standard protein and at least one chaotropic agent further comprises phosphate.

32. Method according to any one of items 25-31, wherein the step of removing residual liquid from said solution comprises removing liquid by means of reduced pressure.

33. Method according to item 32, wherein the step of removing liquid by means of reduced pressure is by means of vacuum drying.

34. Method according to any one of items 32-33, wherein the step of removing liquid by means of vacuum is at a temperature of 5-60° C., such as at 10-50° C., such as at 15-45° C., such as at 20-45° C., such as at 25-45° C., such as at 30-45° C., such as at 35-45° C., such as 40-45° C., such as at 42° C.

35. Method according to item 32, wherein the step of removing liquid by means of reduced pressure is by means of freeze drying.

36. Method according to any one of items 25-35, wherein said method provides retained stability of said at least one internal standard protein upon storage for at least 1 week, such as at least 2 weeks, such as at least 3 weeks, such as at least 4 weeks, such as at least 5 weeks, such as at least 6 weeks, such as at least 7 weeks, such as at least 8 weeks, such as at least 9 weeks, such as at least 10 weeks, such as at least 3 months, such as at least 6 months, such as at least 1 year, such as at least 2 years.

37. Method according to any one of the items 25-36, wherein said method provides retained stability of said at least one internal standard protein upon storage at a temperature of at least 4° C., such as at least 7° C., such as at least 10° C., such as at least 15° C., such as at least 20° C., such as at least 25° C., such as at least 30° C., such as at least 35° C., such as at least 40° C.

38. Method according to any one of items 25-37, wherein said method provides retained stability of said at least one internal standard protein upon storage at a temperature of at most 0° C., such as stored at at most −10° C., such as stored at at most −20° C., such as stored at at most −50° C., such as stored at at most −80° C.

39. Method according to any one of items 25-38, wherein said method provides retained stability of said at least one internal standard protein when subjected to at least 1 freeze-thaw cycle, such as at least 2 freeze-thaw cycles, such as at least 3 freeze-thaw cycles, such as at least 4 freeze-thaw cycles, such as at least 5 freeze-thaw cycles, such as at least 6 freeze-thaw cycles, such as at least 7 freeze-thaw cycles, such as at least 8 freeze-thaw cycles, such as at least 9 freeze-thaw cycles, such as at least 10 freeze-thaw cycles, such as at least 15 freeze-thaw cycles, such as at least 20 freeze-thaw cycles, such as at least 50 freeze-thaw cycles.

40. Method according to any one of items 37-39, wherein said retained stability is determined by a coefficient of variation.

41. Method according to any one of items 25-40, wherein said at least one internal standard protein is at least 2 standard proteins, such as at least 5 standard proteins, such as at least 10 standard proteins, such as at least 20 standard proteins, such as at least 30 standard proteins, such as at least 40 standard proteins, such as at least 50 standard proteins, such as at least 60 standard proteins, such as at least 70 standard proteins, such as at least 80 standard proteins, such as at least 90 standard proteins, such as at least 100 standard proteins, such as at least 200 standard proteins, such as at least 300 standard proteins, such as at least 400 standard proteins, such as at least 500 standard proteins.

42. Method according to any one of items 25-41, wherein said internal standard protein comprises an isotopic label.

43. Method according to any one of items 25-42, wherein said internal standard protein comprises at least one isotopically labeled amino acid.

44. Method according to any one of items 42-43, wherein said isotopic label is selected from the group consisting of ¹⁵N, ¹³C and ¹⁸O.

45. Method according to any one of items 25-44, wherein said internal standard protein is a recombinant protein.

46. Method according to any one of items 25-45, wherein said internal standard protein is a synthetic protein.

47. Container comprising a solid mixture according to any one of items 1-18.

48. Container comprising a solid mixture according to any one of items 19-22.

49. Container obtainable by a method according to any one of items 25-46.

50. Container according to any one of items 47-49, which is selected from the group consisting of a microtiter plate, a vial, a collection tube, a bottle, a pre-coated filter paper, a blood tube, a Whatman paper, a DBS collection device, a dried plasma spot device, a dried serum spot device and a culturing plate.

51. Container according to any one of items 47-50, for use in mass spectrometry.

52. Container according to any one of items 47-51, for use in proteomics.

53. Method for determining the amount of a target protein present in a sample, the method comprising:
providing a container according to item 47;
adding a sample suspected of comprising at least one target protein to said mixture, thereby preparing a test sample,
subjecting said test sample to analysis,
using the results of the analysis to determine the amount of said at least one target protein in said sample by comparison with said internal standard protein.

54. Method for determining the amount of a target protein present in a sample, the method comprising:
providing a container according to item 48;
subjecting said sample to analysis,
using the results of the analysis to determine the amount of said at least one target protein in said sample by comparison with said internal standard protein.

55. Method for determining the amount of a target protein present in a sample, the method comprising:
providing a container according to item 49;
unless already present, adding a sample suspected of comprising at least one target protein to said solid mixture, thereby preparing a test sample,
subjecting said test sample to analysis,
using the results of the analysis to determine the amount of said at least one target protein in said sample by comparison with said internal standard protein.

56. Method according to any one of items 51-54, wherein said internal standard protein comprises a fragment of said target protein.

57. Method according to any one of items 51-56, wherein said analysis is performed using mass spectrometry.

58. Method according to any one of items 51-57, wherein the method further comprises removing residual liquid from said sample.

59. Method according to any one of items 51-58, wherein the method further comprises a step of long-term storage of said sample preceding the steps of subjecting said sample to analysis and determining the amount of said at least one target protein in said sample by comparison with said standard protein.

60. Method according to item 59, wherein said long-term storage is for at least 1 week, such as at least 2 weeks, such as at least 3 weeks, such as at least 4 weeks, such as at least 5 weeks, such as at least 6 weeks, such as at least 7 weeks, such as at least 8 weeks, such as at least 9 weeks, such as at least 10 weeks, such as at least 3 months, such as at least 6 months, such as at least 1 year, such as at least 2 years.

61. Method according to any one of items 53-60, wherein said sample is a bodily fluid sample selected from the group consisting of plasma, serum, blood, cerebrospinal fluid, dry blood spots, saliva and urine.

62. Kit for carrying out the method according to any one of items 53-61, the kit comprising:
a container according to any one of items 47-52, and
instructions for carrying out the method.

1. Solid mixture comprising:
at least one internal standard protein;
at least one chaotropic agent or a derivative or salt thereof; and
optionally a buffer.

2. Solid mixture according to claim 1, wherein said chaotropic agent is selected from the group consisting of urea, guanidine, thiourea and derivatives and salts thereof.

3. Solid mixture according to claim 1, further comprising a sample suspected to comprise at least one target protein.

4. Solid mixture according to claim 3, wherein said internal standard protein comprises a fragment of said target protein.

5. Method for preparing a container comprising a solid mixture comprising at least one internal standard protein, the method comprising:
providing a solution comprising said at least one internal standard protein and at least one chaotropic agent selected from the group consisting of urea, guanidine and derivatives and salts thereof,
placing said solution in a container,
removing residual liquid from said solution,
thereby obtaining a container comprising a solid mixture comprising said at least one internal standard protein and said at least one chaotropic agent.

6. Method according to claim 5, wherein said chaotropic agent is selected from the group consisting of urea, guanidine, thiourea and derivatives and salts thereof.

7. Method according to claim 5, wherein said chaotropic agent is present in said solution in a concentration of at least 0.5 M, such as at least 1 M, such as at least 2 M, such as at least 3 M, such as at least 4 M, such as at least 5 M, such as at least 6 M, such as at least 7 M, such as at least 8 M.

8. Method according to claim 5, wherein the step of removing residual liquid from said solution comprises removing liquid by means of reduced pressure.

9. Container comprising a solid mixture according to claim 1.

10. Method for determining the amount of a target protein present in a sample, the method comprising:
providing a container according to claim 9;
unless already present, adding a sample suspected of comprising at least one target protein to said solid mixture, thereby preparing a test sample,
subjecting said test sample to analysis,
using the results of the analysis to determine the amount of said at least one target protein in said sample by comparison with said internal standard protein.

11. Method according to claim 10, wherein said internal standard protein comprises a fragment of said target protein.

12. Method according to claim 10, wherein the method further comprises a step of long-term storage of said sample preceding the steps of subjecting said sample to analysis and determining the amount of said at least one target protein in said sample by comparison with said standard protein.

13. Method according to claim 10, wherein said sample is a bodily fluid sample selected from the group consisting of plasma, serum, blood, cerebrospinal fluid, dry blood spots, saliva and urine.

14. Kit for carrying out the method according to claim 10, the kit comprising:
a container according to claim 9, and
instructions for carrying out the method.

15. Container comprising a solid mixture obtainable by a method according to claim 5.

16. Method for determining the amount of a target protein present in a sample, the method comprising:
providing a container according to claim 15;
unless already present, adding a sample suspected of comprising at least one target protein to said solid mixture, thereby preparing a test sample,
subjecting said test sample to analysis,
using the results of the analysis to determine the amount of said at least one target protein in said sample by comparison with said internal standard protein.

17. Method according to claim 16, wherein said internal standard protein comprises a fragment of said target protein.

18. Method according to claim 16, wherein the method further comprises a step of long-term storage of said sample preceding the steps of subjecting said sample to analysis and determining the amount of said at least one target protein in said sample by comparison with said standard protein.

19. Method according to claim 16, wherein said sample is a bodily fluid sample selected from the group consisting of plasma, serum, blood, cerebrospinal fluid, dry blood spots, saliva and urine.

20. Kit for carrying out the method according to claim 16, the kit comprising:
a container according to claim 15, and
instructions for carrying out the method.


SEQUENCE LISTING (1 sequence)

SEQ ID NO: 1 - protein, 11 aa, Artificial Sequence
  Engineered quantification peptide for mass spectrometry
DLQAQVVESAK